(19) United States
(12) Patent Application Publication
(10) Pub. No.: US 2012/0274442 A1
(43) Pub. Date: Nov. 1, 2012

(54) METHOD AND SYSTEM FOR MEASURING THE MOBILITY OF AN ANIMAL

(75) Inventor: Toby Mottram, Glasgow (GB)

(73) Assignee: eCow Limited, Glasgow (GB)

(21) Appl. No.: 13/505,393

(22) PCT Filed: Nov. 2, 2010
(86) PCT No.: PCT/GB2010/002025

§ 371 Date: Jul. 16, 2012

(30) Foreign Application Priority Data
Nov. 2, 2009 (GB) 0919162.8

(51) Int. Cl. G06F 7/04

(52) U.S. Cl. 340/5.8

(57) ABSTRACT

A method and system to measure and record the mobility score of quadrupeds is disclosed. The method and system determines when an animal is walking and measures dynamic parameters to assess gait and derive a mobility score.

The present invention relates to assessing the mobility of an animal, and, more specifically, to remotely measuring the mobility score of a quadruped, for example a dairy cow.

Cattle and other quadrupeds (horses, goats, sheep etc) are prone to diseases and conditions of the feet and legs such as lameness that can cause them to have reduced ability to walk without pain or inhibition of gait. Methods exist to assess mobility scores based on human observation of animals as they walk. Such mobility scores are sometimes used to intervene to resolve a lameness issue, but the most usual application is to ensure that farms meet appropriate welfare standards.

Mobility scores assessed by human assessors are routinely used by farmers, veterinarians and welfare assessors to visually assess the mobility of a walking animal on a scale of 0 to 4. A mobility score of 0 represents an animal with no impairment of mobility, while a mobility score of 4 represents an animal being barely able to walk. Mobility scores of 2.5 and above are regarded as indicative of serious and debilitating pain.

Animals with scores above 1.5 should be checked for the development of lesions. However, as herds have become very large, it is difficult to assess the mobility score of every animal with sufficient frequency to detect problems at an early stage. Furthermore, the method of assessing mobility by human assessors is variable between and within assessors and difficult to calibrate to ensure that a common standard is applied between farms.

Radio Frequency Identification Devices (RFID) are widely used to identify animals. Indeed such devices are mandatory in many jurisdictions to identify each animal within national record systems. These RFID are usually embedded within an ear tag and permanently attached to the animal.

More recently, systems have been developed to remotely asses the mobility of animals using sensors. Typically, sensors are attached to the animals' legs and necks. Neck collars are preferable to leg mounted devices as a platform for wireless sensor nodes. This is because the body of an animal attenuates radio signals, particularly in the GHz frequency range, and leg mounted devices have difficulty transmitting to base stations. Therefore, radio transmission from a collar is easier as the transmitting devices or antennae mounted in a collar are likely to be in direct line of sight to base stations.

Furthermore, leg mounted devices are inherently dirty being in the faecal deposit zone. In addition, operator safety is poor for leg sensors as the animal may kick during attachment or adjustment. Leg sensors also have a tendency to rotate around the leg and this may affect the signals from the sensor.

A method for remote detection of lameness is disclosed in GB2437250A/WO2007119070A1. According to this disclosure, an analysis of signals from a neck based accelerometer could be used to determine sharp inflexions in an otherwise sinusoidal trace. A similar method using leg based sensors has been described by Pastell and by de Mol. (Proceedings of the ECPLF Conference, Wageningen, NL, 2009) although no results seem to have been incorporated into a practical system.

Neither of these methods make any distinction between voluntary walking when the animal proceeds at its own speed and induced walking where a herdsman causes the animal to hurry. A lame animal will usually attempt to minimise pain by moving slowing and placing its feet carefully when it moves at its own speed. However, when the animal is hurried, the motion of the neck becomes jerky. Accordingly, there is a need for an automatic monitor to distinguish between voluntary walking and induced walking.

Accelerometers have traditionally been used to record behavioural or long duration events in cattle such as standing, lying and eating. Such events only require relatively slow speed sampling in the range 1-10 Hz, The reason for the low sampling rate is that the available power needed for recording, storing and analysing data is limited on a collar based system. High speed sampling quickly depletes batteries. Accordingly, a mobility monitoring system using accelerometers should only use high speed analysis when the animal is walking. Therefore, accurate indications of when the animal starts and stops walking are required.

Other methods of assessing mobility use video imaging to determine gait parameters (biometrical states). Gait parameters known in the art can be measured and recorded either manually or with image processing techniques applied to images from a camera pointed at an animal laterally when it is walking. Such gait parameters can be derived from video imaging using various algorithms, however this requires animals to walk in single file past a fixed camera position with sufficient depth of view to capture good images. Furthermore, sunshine may create strong contrasts which affect the ability of algorithms to detect the shape of animals' legs in the image. Accordingly, if gait parameters are used in assessing mobility, there is a need to reduce the effect of lighting changes on the images obtained.

In summary, the existing methods for assessing mobility described above do not adequately provide routine lameness detection. The first requirement for an effective system for measuring mobility is to determine precisely when the animal is voluntarily walking and for how long, recording data about mobility over a long period to remove the day to day variability. Improved precision in determining the mobility score can be achieved by electronic devices attached to the animal that identify accelerations that relate to mobility.

The present invention was devised to address all of the above requirements in order to provide a complete system for objectively measuring mobility scores of cattle or other quadrupeds. More specifically, the present invention is aimed at remotely measuring the mobility score of a quadruped using an array of fixed devices and sensors, animal mounted devices and sensors and an analytical database.

According to the present invention, there is provided a method for measuring the mobility score of an animal, the method comprising the steps of:
detecting the animal walking in a mobility sensing zone;
determining the identity of the walking animal;
determining the time taken by the identified animal to walk through the mobility sensing zone;
storing the determined time in an individual record for the identified animal in a database;
repeating the steps of detecting the animal, determining the identity,
determining the time and storing the determined time; and
calculating the mobility score based on the individual record.

According to the present invention, there is also provided a system for measuring the mobility score of an animal, the system comprising:
a mobility sensing zone;
means for detecting the animal walking in the mobility sensing zone;
means for determining the identity of the walking animal;
means for determining the time taken by the identified animal to walk through the mobility sensing zone;
a database for storing the determined time in an individual record for the identified animal; and
a central processor for calculating the mobility score based on the individual record.

According to the present invention, the analysis of the time taken by the animal to walk through the mobility sensing zone, or time of flight (TOF) is sufficient to determine mobility scores which correlate well with those determined by human assessors.

Preferably, an identification device such an RFID ear tag is mounted on the animal and the means for detecting the animal walking comprise a wireless system for wirelessly sensing the identification device. Thus, existing RFID ear tags which are normally used solely for identification, can be used to determine the TOF and mobility scores.

A method according to the present invention may further comprise the steps of sensing data by a sensing device mounted on the identified animal and recording the sensed data in the individual record, wherein the step of calculating comprises performing a statistical analysis of the data recorded in the database. Preferably, the sensing device is a sensing collar comprising at least one accelerometer and the sensed data includes acceleration data in one, two or three dimensions. Statistical analysis may include at least one of determining curtosis, using Kalman filters, determining peak accelerations in each of the three directions, identifying asymmetry in movement patterns and performing Fast Fourier Transform analysis. Accordingly, with the present invention, improved precision in determining the mobility score can be achieved by electronic devices attached to the animal that identify accelerations which relate to mobility.

Preferably, data is recorded at a frequency rate of 1-250 Hz whilst the animal is walking in the mobility sensing zone. Thus, battery life is increased by only using high speed sampling when the animal is walking.

According to the present invention, walking in the mobility sensing zone may comprise walking in a sensing passageway provided with a camera for obtaining images of the walking animal and the method may further comprise the steps of: obtaining at least one image of the animal as it walks through the passageway, determining at least one gait parameter from the at least one image; storing the at least one gait parameter in the individual record; repeating the steps of obtaining at least one image, determining at least one gait parameter and storing at least one gait parameter. In this embodiment, calculating the mobility score may be based on the at least one gait parameter to further improve precision in determining the mobility score.

Preferably, the sensing passageway comprises means for controlling the walking of the animal and means for controlling the lighting. This allows for a more precise dynamic image analysis which can be enhanced by controlling the movement of the animals and the light falling on the animals legs when images are captured. Controlling the lighting reduces the variability between images and simplifies the processing.

Preferably, the camera obtains lateral images of the walking animal to enable assessment of standard gait parameters.

Accordingly, the present invention uses a hierarchy of measurements that implemented at the minimal level establish the state of the mobility of an animal at the fullest level and can indicate the degree of lameness of each foot. The mobility sensing zone is of variable size but the system comprises various sensors and means to collect, store and process data from the sensors.

Therefore, the present invention provides a method and system for routine lameness detection that is based on objective sensing and that is repeatable between animals.

Examples of the present invention will be described with respect to the following drawings, in which:

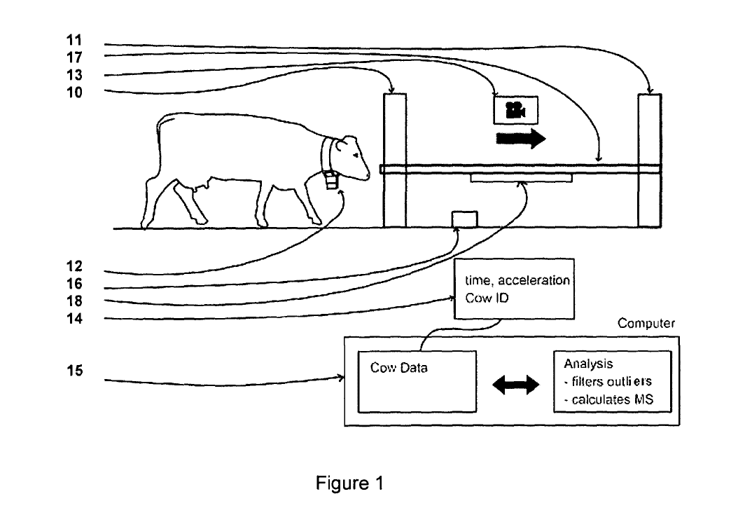

FIG. 1 is a schematic representation of a mobility sensing system according to the present invention;

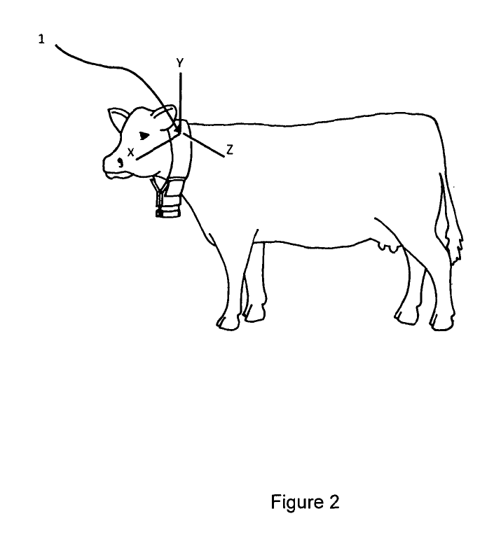

FIG. 2 shows the placement and orientation of accelerometers on a cow's collar;

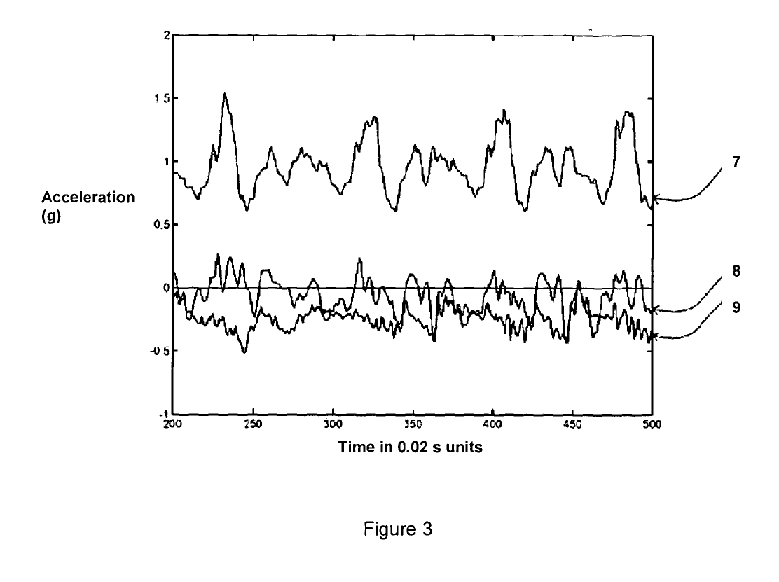

FIG. 3 shows types of signal recorded from neck mounted accelerometers; and

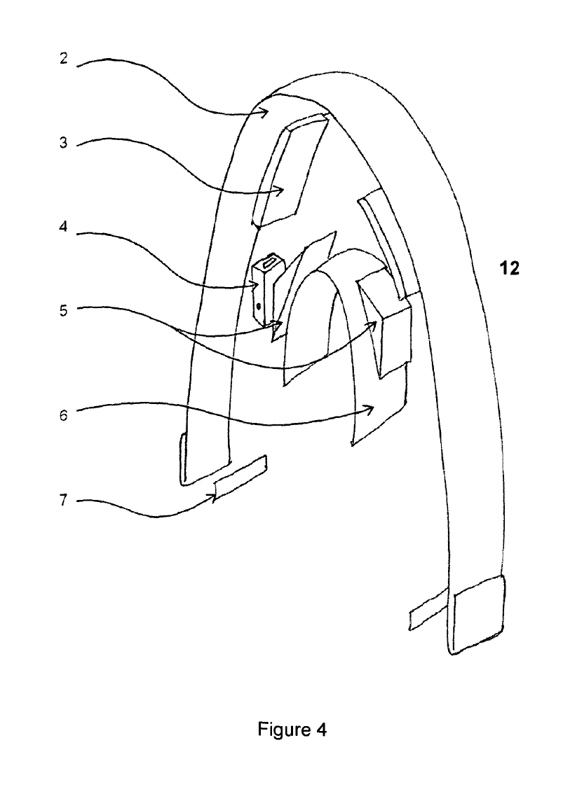

FIG. 4 shows a sensing collar for measuring accelerations on a cow's neck.

FIG. 1 is a schematic representation of the mobility sensing system and data flow according to the present invention. A wireless system (not shown) may comprise sensors and radio antennas, directional radio antennas, or RFID antennas which are located at the start point 10 and end point 11 of a mobility sensing zone 17 to detect the passage of the animal through the zone 17.

In the case of dairy cows, the recommended place for the positioning of the mobility sensing zone 17 is in the area that the animal enters on leaving the milking parlour. Dairy cows are usually milked once or more times per day. When a cow leaves a milking parlour, it often moves at its natural pace. The exit passage of the milking parlour is therefore a suitable place to asses the mobility of cows.

Furthermore, the exit passage of the milking parlour has the advantage that it provides a mobility sensing zone 17 unlikely to obstruct the animal's progress, with clearance occurring approximately every 20 s. This avoids a scenario where several animals may be in the sensing zone 17 at any one time. In this scenario, if the mobility sensing zone 17 is sufficiently wide, more mobile animals may overtake the less mobile ones. Furthermore, in a narrow sensing zone, slow animals may restrict those behind. Therefore, when several animals are in the mobility sensing zone, suitable software analysis is required to identify and remove these effects.

The detection of the cow in the milking parlour may be achieved by using RFID such as RFID ear tags or other automatic means of identification. When the cow is released from the milking parlour either individually or in a group, the time is recorded by a sensing module (not shown). The sensing module may be preferably fitted in the animal's collar such as a sensing collar 12. At the time when the animal passes the end point 11, the sensing module records this time and then calculates the time taken to pass through the sensing zone 17 or TOF. The shorter the sensing zone 17, the higher the accuracy of the time recording required, typically milliseconds.

The start sensor at the start point 10 of the mobility sensing zone 17 may thus be a simple microswitch whose contacts open when the parlour exit gate is released. The start sensor may also be an RFID antenna receiving a signal transmitted by the RFID ear tag of the cow. Alternatively, the start sensor at the start point 10 of the mobility sensing zone 17 may be incorporated into a mechanical gate or turnstile that allows only one animal to enter part or all of the mobility sensing zone 17 at a time.

A record for each animal is created by the sensing module which stores the animal's unique identity. A wireless system may detect the time when a RFID mounted on the animal such as an RFID ear tag passes the start point 10 of the mobility sensing zone 17. This time represents the start time when the animal enters the zone 17. The wireless system transmits the start time to the sensing module and the start time is then recorded by the sensing module.

The identification of the animal may optionally be achieved by a sensing collar 12. A sensing collar 12 may further contain an electronics package including a microprocessor, a wireless link and an accelerometer for measuring accelerations on the animal.

As disclosed in GB2447101A, collar systems have been developed that are easy to attach and stay in a steady relative position on the animal. Collars permit a comfortable load that can be up to 2% of body weight in accordance with traditional loads such as cow bells and ox yokes. Practical collar devices with weights up to 700 g appear to be optimal for cow comfort and stability on the neck. A collar of this weight permits battery packs that allow long operation of electronics.

Ear tag mounting of sensors is also an option for measuring accelerations but, has the disadvantage that the device needs to be of low weight (below 70 g) to allow secure attachment. Furthermore, ear accelerations are less closely correlated to the cow's gait as the cow uses its ears for directional audition and insect control and these movements may confound gait measurements.

FIG. 2 shows an accelerometer mounted on a neck collar 1 with three axes (X, Y, Z) of measurement, of the type which can be used in a system according to the present invention. The accelerometer records forward acceleration in the X axis direction and head raising and lowering along the Y axis on which there is a constant acceleration of 1 g due to gravity. Turning movements are mainly detected as a change in signals detected on the Z axis.

In conventional motion detection systems, sensors can be mounted on containers and vehicles in guaranteed alignment to the plane of the ground.

Unfortunately, an animal has no flat surfaces and there is no guarantee that it will be in alignment with any reference point. However, sensing collars as shown in FIG. 2 provide for a cow in the standard anatomical position an approximate alignment of the Y axis perpendicular to the plane of the earth surface and an approximate alignment of the X and Z axes parallel to the plane of the earth surface.

A walking quadruped normally has three feet on the ground at any one instant in time. The feet move in a sequence with the rear foot pushing forward followed by the front on the same side. Accelerations in the X, or forward direction, are associated with the hind leg propulsion movement. A peak in the X acceleration occurs as the animal begins to thrust forward and is always matched by later deceleration but not at the same rate. However, the mass and inertia of the animal constrain the size of accelerations in the X axis. The Y axis, or vertical acceleration, indicates the movement of the head vertically. The Y axis accelerations are generally larger than those in the X axis and can be up to 7 g for an animal which jumps and is clearly not in any pain.

With accelerometers mounted on sensing collars of the type shown in FIG. 2 it is possible to create graphs such as that shown in FIG. 3. FIG. 3 shows typical accelerations in X (8), Y (7) and Z (9) sampled at 50 Hz as a function of time for a cow walking for 6 seconds. The Y accelerations 7 indicate that the neck oscillates vertically at approximately 1 g with a regular pattern repeating at approximately 2 s intervals. The X accelerations 8 show that the forward accelerations of the cow oscillate about 0. Changes in the relationships between the peak accelerations in the X and Y directions can be used to determine the likelihood of one or other rear leg being lame. The Z accelerations 9 show few changes as the cow walks in a straight line.

FIG. 4 shows a sensing collar 12 for measuring accelerations on animal's neck, of the type which can be used in a system according to the present invention. The sensing collar 12 is provided with a webbing 2 of polyester or similar material 50-70 mm wide and approximately 1.5 m in length to which a protective foam element 3 is attached by stitching or glue. A wedge shaped piece 5 is used to enable an approximate alignment with the vertical (perpendicular to the plane of the earth surface) of electronics packages mounted on the collar, including an accelerometer 4. On the contralateral of the cow a matching shape is mounted to provide balance. The whole assembly is secured under the webbing 2 with a polypropylene or other material piece 6 sewn or glued to create a yoke. The inward facing side of the yoke 6 fits into the concavity at contralateral to the dorsal part of the neck when the head of the animal is up.

The shape and positioning of the electronics packages in the sensing collar 12 are important to ensure consistent sensor positions on the animal. When the head is down, the soft fabric allows the collar to fit the now fattened neck, the alignment changes so that the Y axis is approximately parallel to the ground plane and the Z axis vertical to it. However, these complex movements can be ignored if it can be identified that the animal is walking and consequently that its head is up to provide forward vision.

An electronics package in the sensing collar 12 may include an accelerometer 5 with 1-3 axes, a movement detection sensor, a microprocessor with timing and memory, batteries and signal transceiving software. The electronics package also includes a receiving device and a transmitting device. The receiving device is included to activate the start of sampling. Each receiving device has a unique identity provided either by RFID or an included unique identification means maintained by the microprocessor and linked to the animal's unique identity stored by the sensing module.

The passing of the start point 10 triggers the start of high-speed sampling at frequencies between 1 and 250 Hz. High speed sampling is capable of identifying accelerations up to 10 g.

This high rate of sampling is only required for short durations from 1-30 seconds when the animal is known to be walking, such as, in the case of dairy cows, at the exit from a milking parlour. Where the sensing collar 12, or other device, is used to detect parameters of animal behaviour other than lameness, the sampling is switched on when the animal enters the mobility sensing zone 17 and switched off when the animal leaves it.

To avoid unnecessary sampling and thus consumption of battery power of the sensing collar 12, the sensing module may be switched on after receiving a command from the database. The sensing module 12 may be switched off at selected points of the animal's passage through the mobility sensing zone 17 or at times during the animal's lactation cycle. For example, in the case of dairy cows, sampling may occur only during morning/evening milkings or only a few days in any month.

The passing of the end point 11 may trigger reverting to normal speed sampling or quiescence. As the animal passes the end point 11, calculated parameters, including times, velocity values, acceleration values as well as parameters stored by the sensing collar 12 are transmitted to a radio antenna of a wireless system (not shown) for processing by a central processor (not shown) or system computer. All of the above values may be stored in an individual record in a database.

In one embodiment of the present invention a sensing passageway 18 (also shown in FIG. 1) is included within the mobility sensing zone 17. As the animal passes through the entry point 10 of the mobility sensing zone 17 an imaging camera 19 pointed laterally at the animal may be activated. As the animal passes through the sensing passageway 18 the imaging camera records images of the animal walking.

Images of the animal's gait may be analysed by an image processing unit 14. The biomechanical features of gait that can be measured with the image processing unit 14 are defined below.
1. Trackway Overlap is the difference between the grounded position of foreleg and hindleg. An animal places its hind foot in the position just vacated by the front foot of the same side. A cow with gait score 0 will have a Trackway Overlap close to 0. With some types of lameness the cow will not be able to place the foot in that position and the Trackway Overlap will be greater than 0.
2. Stride Length is the difference between the position occupied in successive steps of the same foot. A cow with gait score 0 has a stride length longer than those of cows with higher scores.
3. Swing Time is the time that a foot is not in contact with the ground.
4. Stance Time is the time for which the hoof is in contact with the ground.
5. Touch Angle is the angle of the fetlock joint when the animal touches its foot down. Depending on the state of the foot and the pain that any lesions cause the animal the angle will change from higher angles with gait score 0 to almost vertical where the animal tends to minimise pain in its foot.
6. Release Angle is the angle of the fetlock to the ground as the animal pushes off from the foot. This is affected by the pain status of the foot or leg.

The image processing unit 14 may determine one or more of the six gait parameters defined above. The determined gait parameters are then transmitted to the sensing module and linked to the animal's identity by matching of time of start and stored for later retrieval.

The optional sensing passageway 18 is a structure which permits an animal to enter alone and take one or more complete cycles of steps of all legs. A length of the passageway 18 of over 2.5 m is normally required. The floor of the passageway 18 can optionally have obstructions 16 to cause the animals to alter their gait. The obstructions may consist of one or more of a step up, a step down, a ramp up, ramp down, and a round bar set at any height up to 0.5 m above the floor. The purpose of the obstruction is included to cause the animal to adjust its gait and thereby alter the accelerations recorded on its neck in a predictable way.

The sides and roof of the passageway 18 may be of translucent material to diffuse light and prevent shadows caused by direct sunlight or artificial light. The passageway 18 is equipped with lights to illuminate the legs of the animal and permit good images to be collected by a camera 19 mounted laterally. The passageway 18 may be curved to create a continuous focal length for the laterally mounted camera 19.

The entry point of the sensing passageway 18 may also be the start point of the mobility sensing zone 11, while the exit post of the sensing passageway 18 may also be the end point of the complete mobility sensing zone 11. Instrumented posts at the start 10 and end 11 points of the mobility sensing zone 17 may be used to activate and de-activate the sensing systems as the animal enters and leaves the sensing passageway 18. The entry and exit in the passageway 18 may be detected by a RFID device (not shown) or by another wireless device mounted in the sensing collar 12 or elsewhere on the animal. Due to latency in radio signal transmission and decoding, an optional optical sensor may be used to identify the precise time when the animal passes the start 10 and end 11 points of the mobility sensing zone 17.

The sensing passageway 18 ensures that individual animals are presented to the sensors in a controlled manner both in speed and ambient conditions. As the animal passes the entry of the sensing passageway 18 the animal is identified and a period of high speed recording is initiated by the sensing module, If required, the imaging camera 19 may be switched on.

The wireless system at the entry post 10 has a narrow angle of reception, or uses a directional antenna, so that the start of high speed recording is limited only to the animal entering the passageway 18. As the animal leaves the passageway 18 it passes an exit post 11 also equipped with a wireless system or directional antenna that records the identity of the animal and causes the electronics in the sensing collar 12 to revert to slow speed monitoring.

The data received by the sensing module may be analysed by the sensing module to determine the mobility score. The data includes TOF and optionally, accelerations. Optionally, the data may further include gait parameters derivede from images.

The data recorded and/or analysed by the sensing module during the period whilst the animal is passing through the passageway 18 or the complete mobility sensing zone 17 is wirelessly transmitted to an antenna at the exit post 11. The mobility score and any other data may be then recorded in individual records of a database so that changes over time for an identified animal can be identified and reported via a computer. The sensing module, data analysis and flow are described in more detail below.

The sensing module is a piece of software that may run on an independent computing platform or may be combined with other software on a herd database.

The purpose of the sensing module is to organise, compute and store data from the mobility sensing zone 17, the sensing collar 12 and the sensing passageway 18. The sensing module may calculate a mobility score by combining some or all of the data recorded and may transmit the mobility score and some or all of the measured parameters to a database 15 for regular analysis to measure changes in parameters as animals suffer or recover from lameness. The minimum amount of data stored in the individual record for each animal is the time taken to walk between the start 10 and end 11 point of the mobility sensing zone 17.

The time taken to pass through the mobility sensing zone 17 and the time taken to pass through the sensing passageway 18 are correlated to the mobility score. The mobility measurement system records the time taken for an animal to pass from the start point 10 to the end point 11 of the mobility sensing zone 17. This time may be recorded for example at each milking and stored in an individual record or longitudinal file for each animal.

The time taken will depend on the length of the installed mobility sensing zone 17. A mobility sensing zone 17 of 20 m is normally traversed by a cow with mobility 0 in about 4 s whilst a cow of score 3 may take 20 s. There will be variability due to variations in the day to day behaviour of the animal and occasional agonistic interactions with other animals but over a period the time taken to traverse the mobility sensing zone 17 approach a mean. The time taken will inversely correlate to the mobility score and it is sufficient to asses the mobility score.

The individual record is created for each animal and includes at least times taken by the animal to pass through the mobility sensing zone. Deviations from the data stored in the individual record may be indicative of increasing mobility scores. For example, if the time taken by an animal begins to increase over the mean value, it is likely that the mobility score is increasing and the animal is becoming lame. A message will be generated by the database 15 when the mobility score changes by more than the standard deviation of the time series. The farmer or veterinarian can inspect the data for individual animals.

Where a collar sensing system 12 is used the amount of data stored could be as much as 30 s recorded for 3 axes of accelerations and can be processed mathematically to determine gait characteristics that can be correlated to mobility score with deterministic signal processing.

The features of the accelerations in X, Y and Z directions are characterised by one or more parameters, including curtosis (kurtosis). The curtosis of the values of acceleration on each axis of movement is lower for lame animals. The maximum values of acceleration of axis of movement are inversely correlated to mobility score. For example, when a cow is driven from pasture, its motion is jerky as the cow is presumably responding to pain in the feet giving high curtosis values particularly of the Y axis. However, when not driven, as in the mobility sensing zone 17, lame cows walk slowly with minimal movements to minimise pain.

More detailed signal processing can extract further information. Further analysis of the mean, the standard deviation, the median and the number of peak values of accelerations in each axis of movement can be used to improve correlations.

Signals in the X and Y axes can be resolved into waveforms generally of period 1-2 seconds. The Y axis of FIG. 2 shows a typical waveform. Waveforms are of short duration with rarely more than six cycles unless the length of the mobility zone 17 is extended. As well as curtosis, standard signal processing techniques such as Kalman filters, or peak accelerations in the X, Y, or Z directions, or irregularity/asymmetry in movement pattern, Fast Fourier Transform analysis, or a combination of several of these techniques can be used to characterise the waveforms and correlate them to mobility score.

If the sensing passageway 18 is used to collect image data, the image processing unit 14 extracts one or more of the six gait parameters defined above and transfers these to the sensing module.

In recent years, collar mounted wireless sensor systems have been introduced to detect changes in cow behaviour related to oestrus and to calving. However, none of these systems detect lameness. The present invention could be used to extend the existing systems to include the measurement of cow lameness.

1. A method for measuring the mobility score of an animal, the method comprising the steps of:
detecting the animal walking in a mobility sensing zone;
determining the identity of the walking animal;
determining the time taken by the identified animal to walk through the mobility sensing zone;
storing the determined time in an individual record for the identified animal in a database;
repeating the steps of detecting the animal, determining the identity, determining the time and storing the determined time; and
calculating the mobility score based on the individual record.

2. A method according to claim 1, wherein detecting the animal walking in the mobility sensing zone comprises wirelessly sensing an identification device mounted on the animal.

3. A method according to claim 2, wherein the identification device is an RFID ear tag.

4. A method according to claim 3, further comprising the steps of:
sensing data by a sensing device mounted on the identified animal; and
recording the sensed data in the individual record,
wherein the step of calculating comprises performing a statistical analysis of the data recorded in the database.

5. A method according to claim 4, wherein the sensing device is a sensing collar comprising at least one accelerometer and wherein the sensed data includes acceleration data in one, two or three dimensions.

6. A method according to claim 5, wherein the statistical analysis includes at least one of determining curtosis, using Kalman filters, determining peak accelerations in each of the three directions, identifying asymmetry in movement patterns and performing Fast Fourier Transform analysis.

7. A method according to claims 6, wherein the data is recorded at a frequency rate of 1-250 Hz whilst the animal is walking in the mobility sensing zone.

8. A method according to claim 7, wherein walking in the mobility sensing zone comprises walking in a sensing passageway provided with a camera for obtaining images of the walking animal, the method further comprising the steps of:
obtaining at least one image of the animal as it walks through the passageway;
determining at least one gait parameter from the at least one image;
storing the at least one gait parameter in the individual record;
repeating the steps of obtaining at least one image, determining at least one gait parameter and storing at least one gait parameter.

9. A method according to claim 8, wherein said step of calculating the mobility score is based on the at least one gait parameter.

10. A method according to claim 9, wherein the sensing passageway comprises means for controlling the walking of the animal.

11. A method according to claims 10, wherein the sensing passageway comprises means for controlling the lighting.

12. A method according to claims 11, wherein the camera obtains lateral images of the walking animal.

13. A system for measuring the mobility score of an animal, the system comprising:
a mobility sensing zone;
means for detecting the animal walking in the mobility sensing zone;
means for determining the identity of the walking animal;
means for determining the time taken by the identified animal to walk through the mobility sensing zone;
a database for storing the determined time in an individual record for the identified animal; and
a central processor for calculating the mobility score based on the individual record.

14. A system according to claim 13, wherein an identification device is mounted on the animal and wherein the means for detecting the animal walking comprises a wireless system for wirelessly sensing the identification device.

15. A system according to claim 14, wherein the identification device is an RFID ear tag.

16. A method for measuring the mobility score of an animal, the method comprising the steps of:
detecting the animal walking in a mobility sensing zone by wirelessly sensing an identification device mounted on the animal, said mobility sensing zone comprising a sensing passageway provided with a camera for obtaining images of the walking animal;
determining the identity of the walking animal;
determining the time taken by the identified animal to walk through the mobility sensing zone;
storing the determined time in an individual record for the identified animal in a database;
repeating the steps of detecting the animal, determining the identity, determining the time and storing the determined time;
calculating the mobility score based on the individual record;
sensing data by a sensing device mounted on the identified animal;
recording the sensed data in the individual record;
obtaining at least one image of the animal as it walks through the passageway;
determining at least one gait parameter from the at least one image;
storing the at least one gait parameter in the individual record; and
repeating the steps of obtaining at least one image, determining at least one gait parameter and storing at least one gait parameter,
wherein the step of calculating comprises performing a statistical analysis of the data recorded in the database.

17. A method according to claim 16, wherein the identification device is an RFID ear tag, wherein the sensing device is a sensing collar comprising at least one accelerometer, and wherein the sensed data includes acceleration data in one, two or three dimensions.

18. A method according to claim 17, wherein the statistical analysis includes at least one of determining curtosis, using Kalman filters, determining peak accelerations in each of the three directions, identifying asymmetry in movement patterns and performing Fast Fourier Transform analysis.

19. A method according to claim 18, wherein calculating the mobility score is based on the at least one gait parameter.

20. A method according to claim 16, wherein the sensing passageway comprises means for controlling the walking of the animal and means for controlling the lighting, and wherein the camera obtains lateral images of the walking animal.
